(19) United States
(12) Patent Application Publication
(10) Pub. No.: US 2015/0047665 A1
(43) Pub. Date: Feb. 19, 2015

(54) HAIR CARE COMPOSITIONS WITH ENHANCED COLOR RETENTION AND METHODS OF PREPARATION THEREFOR

(71) Applicant: FramKat LP, Leetsdale, PA (US)

(72) Inventors: Thomas A. Lehman, Indianapolis, IN (US); Maria R. Pittavino, Aliquippa, PA (US); Allison Tonk, Carmel, IN (US)

(73) Assignee: FramKat LP, Leetsdale, PA (US)

(21) Appl. No.: 14/462,700

(22) Filed: Aug. 19, 2014

Related U.S. Application Data
(60) Provisional application No. 61/867,474, filed on Aug. 19, 2013.

(51) Int. Cl. A61K 8/97; A61K 8/894; A61Q 5/00; A61K 8/81; A61K 8/58

(52) U.S. Cl. CPC A61K 8/97; A61K 8/8182; A61K 8/8158; A61K 8/585; A61Q 5/004; A61K 8/894; A61K 2800/10
USPC 132/202; 424/70.15; 424/70.122

(57) ABSTRACT

Hair care compositions, such as shampoo and conditioner compositions, for use with color-treated hair. The compositions when used, for example, to shampoo or condition color-treated hair, being effective to protect color-treated hair such that, for example, there is minimal adverse effect on the color of the color-treated hair, such as fading. The compositions include a cationic terpolymer of vinylpyrrolidone, dimethylaminopropyl methacrylamide, and methacryloylaminopropyl lauryldimonium chloride, quinoa and a first silicone. There is also provided a method of imparting color retention characteristics to shampoos and conditioners by formulating the shampoos or conditioners to include a cationic terpolymer of vinylpyrrolidone, dimethylaminopropyl methacrylamide, and methacryloylaminopropyl lauryldimonium chloride, quinoa and a first silicone. Optionally, the hair care compositions can include a second silicone compound.

### CROSS REFERENCE TO RELATED APPLICATIONS

This application claims priority under 35 U.S.C. §119(e) to U.S. Provisional Patent Application Ser. No. 61/867,474, filed Aug. 19, 2013, entitled “Shampoo and Conditioners Having Good Color Retention Characteristics,” which is herein incorporated by reference.

### FIELD OF THE INVENTION

This invention relates generally to hair care compositions and, more particularly, to shampoos and conditioners for dyed or color-treated hair. The shampoos and conditioners provide enhanced effectiveness in retaining the dye or colorant in the hair and protecting the dye or colorant from fading or washing-out.

### BACKGROUND OF THE INVENTION

Various compositions and methods for color treating hair by applying artificial colorant or dye are well known and employed by many women, and also some men. Retaining the as-applied color of the treated hair is a common problem. For instance, as a result of applying colorant or dye, the hair exhibits a particular color and/or shine. However, this color and/or shine exhibited immediately after applying the colorant or a short time thereafter begins to fade or wash-out as a result of applying shampoo and conditioner to the color-treated hair during washing and conditioning. Generally, surfactants which are typically present in many known shampoo and conditioner formulations perform a wetting function that provides moisture into the hair shaft and facilitates removal of the dye molecules during the rinsing process. Thus, the artificial dye applied to the hair in the color treatment process is disrupted and subsequently has a tendency to be removed during washing, rinsing and conditioning of the hair over a period of time.

Retaining artificial dye applied to hair and minimizing color fading are highly desirable features in hair care products. Several anti-fading products are commercially available including anti-fading shampoos and conditioners. Some of these products contain silicones such as dimethicone and amodimethicone, which are believed to improve color retention.

Certain shampoos and conditioners known in the art are formulated for use by commercial salons and the professionals employed by these salons. It is typically believed by the consumer that hair care products targeted for use by the professional salon industry are a special and improved category of shampoos and conditioners as compared to shampoos and conditioners sold “over the counter” directly to the consumer. However, in both categories, e.g., direct-to-consumer and salon professionals, there are a wide variety of shampoo and conditioner products designed, formulated and targeted for use with color-treated hair. These products which are particularly useful for color-treated hair typically contain one or more ingredients that may be effective to enhance the retention of artificial dye which has been applied to color-treated hair. It is a desire and objective of both the consumer and the product manufacturer to retain for as long as possible the initial color exhibited immediately following color treating of the hair. Thus, there is a need in the art for compositions and methods to prevent the artificial dye from washing-out or fading as a result of subsequent washing and conditioning steps applied to color-treated hair.

It is further understood that many of the products that are advertised and sold specifically for color-treated hair are not as effective as desired at color or dye retention. Thus, there is room for improvement and a desire to develop hair care products, such as shampoos and conditioners, that are even more effective at reducing fade and wash-out, and improving color retention.

### SUMMARY OF THE INVENTION

These needs and others which will become apparent from the following description are achieved by the invention. In one aspect, the invention provides a hair care composition including a cationic terpolymer of vinylpyrrolidone, dimethylaminopropyl methacrylamide, and methacryloylaminopropyl lauryldimonium chloride, quinoa and a first silicone.

The color-treated hair has applied thereto an artificial dye and the hair care composition may be effective to at least partially prevent the artificial dye from fading and washing out.

In certain embodiments, the hair care composition is selected from the group consisting of shampoos and conditioners.

The first silicone can be an anionic silicone and in certain embodiments, may be selected from the group consisting of silicone quaternium-16, undeceth-11, butyloctanol, undeceth-5, and mixtures thereof.

The composition further can include a second silicone, such as but not limited to a silicone amine which may include bis-isopropylamino-PG-propyl dimethicone/bis-isobutyl PEG-14 copolymer and polysorbate 20. In certain embodiments, the second silicone includes a mixture of bis-isopropylamino-PG-propyl dimethicone/bis-isobutyl PEG-14 copolymer, polysorbate 20 and butyloctanol.

In certain embodiments, the first silicone includes a mixture of silicone quaternium-16, undeceth-11, butyloctanol, undeceth-5, and the second silicone includes a mixture of bis-isopropylamino-PG-propyl dimethicone/bis-isobutyl PEG-14 copolymer, polysorbate 20 and butyloctanol.

In another aspect, the invention provides a method of preparing a hair care composition for application to color-treated hair, the composition having improved color retention. The method includes combining the following components: a cationic terpolymer of vinylpyrrolidone, dimethylaminopropyl methacrylamide, and methacryloylaminopropyl lauryldimonium chloride, quinoa and a first silicone.

In still another aspect, the invention provides a method of improving the retention of artificial color for color-treated hair during washing and rinsing of the color-treated hair. The method includes preparing a composition by combining the following components: a cationic terpolymer of vinylpyrrolidone, dimethylaminopropyl methacrylamide, and methacryloylaminopropyl lauryldimonium chloride, quinoa and a first silicone; wetting the color-treated hair; applying the composition to wetted color-treated hair and then rinsing with water to at least partially remove the composition from the color-treated hair.

### DETAILED DESCRIPTION OF THE INVENTION

The invention relates generally to compositions for keratin fibers, in particular for human keratin fibers and more specifically, for human hair. The invention also includes methods for preparing the compositions and uses of the compositions for hair care. In particular, the compositions, methods and uses of the invention are directed to hair that is treated with artificial dye or colorant to produce color-treated hair. The application of artificial dye or colorant to human hair is widely known and practiced in the art. For example, there are a wide variety of known oxidation-dyeing compositions for hair which typically include an oxidation dye in a medium suitable for dyeing hair. Further, it is known that subsequent washing and rinsing of the color-treated hair can result in fading and washing out of the artificial dye from the hair. Furthermore, it is known in the art that there is a desire and need to develop hair care products that minimize the extent and effects of fade and wash-out. The hair care compositions, methods and uses of the invention are effective to increase or improve retention of artificial dye in hair even after subjecting the color-treated hair to multiple washing and rinsing steps, as compared to dye or color retention exhibited by known hair care compositions.

The hair care compositions in accordance with the invention include shampoo and conditioner compositions. These compositions are typically applied to wetted hair and subsequently rinsed with water to remove at least a portion of the compositions from the hair. Shampoo compositions are typically employed for cleansing the hair. Conditioner compositions are typically employed to render hair more healthy and manageable. It is known to use these compositions separately or in combination. It is typical for a shampoo composition to be applied and rinsed, followed by the application and rinsing of a conditioner composition.

The hair care compositions according to the invention include a combination of a polymer component, a quinoa component and a silicone component. The polymer component includes a cationic terpolymer of vinylpyrrolidone, dimethylaminopropyl methacrylamide, and methacryloylaminopropyl lauryldimonium chloride. This polymer component is known in the art and is commercially available as Polyquaternium-55. The International Nomenclature for Cosmetic Ingredients (INCI) directory identifies Polyquaternium-55 as N,N-dimethyl-N-[3-(2-methyl-2-propenoyl)aminopropyl]-1-dodecanaminium chloride, polymer with N-(3-dimethylaminopropyl)-2-methyl-2-propenamide and 1-ethenyl-2-pyrrolidinone. The amount of the polymer component, e.g., Polyquaternium-55, present in the shampoos and conditioners according to the invention can vary widely. In certain embodiments, the polymer component, e.g., Polyquaternium-55, can be present in a shampoo or conditioner composition in an amount from greater than zero to about 10%, or from about 0.5% to about 2.50%, or from about 0.5% to about 1.25%, by weight based on total weight of the composition.

The quinoa component of the compositions according to the invention can be selected from those quinoa materials known in the art. In general, quinoa is a grain crop having high protein seeds. Quinoa is grown, harvested and processed using conventional apparatus and techniques. There are a wide variety of processed quinoa materials that are commercially available and suitable for use in the invention. Non-limiting examples of suitable quinoa materials include commercially available products, such as but not limited to, Quinoa Pro Ex which is available from Tri-K and Hydrolyzed Quinoa Protein which is available from Vege Tech. The amount of quinoa present in the shampoos and conditioners according to the invention can vary widely. In certain embodiments, the quinoa can be present in a shampoo or conditioner composition in an amount from greater than zero to about 10%, or from about 0.10% to about 5.0%, or from about 0.25% to about 2.0%, or from about 0.25% to about 0.5%, by weight based on total weight of the composition.

The silicone component can include one or more silicone materials that are known in the art for use in hair care compositions. Further, the compositions according to the invention can include a first silicone component and/or a second silicone component. The first silicone and/or second silicone components can include an anionic silicone. In certain embodiments, the first silicone component includes a one or more polymers having quaternary ammonium functional groups, such as quaternary ammonium salts. A non-limiting example includes a polymer known in the art and commercially available as Silicone Quaternium-16. This material is identified in the International Nomenclature for Cosmetic Ingredients (INCI) directory. Silicone Quaternium-16 is typically used as a surfactant in hair care compositions.

In certain embodiments, the first silicone component includes polyethylene glycol derivatives of undecyl alcohol and/or polyethylene glycol ether of undecyl alcohol, such as 2-undecoxyethanol and polyoxyethylene (11) undecyl ether, respectively, which are known in the art and commercially available as Undeceth-5 and Undeceth-11, respectively. These materials are identified in the International Nomenclature for Cosmetic Ingredients (INCI) directory. It is known in the art to use Undeceth-5 and Undeceth-11 as surfactants in hair care compositions.

In certain embodiments, the first silicone component includes 2-butyloctan-l-ol which is known as butyloctanol. Butyloctanol is known in the art as a solvent and is commercially available from various sources.

A non-limiting example of a suitable first silicone component is DC 5-7113 which is commercially available from Dow Corning and is identified as a 22 percent nonionic emulsion of a cationized aminofunctional silicone polymer for use in hair care compositions which includes a mixture of Silicon Quaternium-16, Undeceth-5, Undeceth-11 and butyloctanol.

The amount of the first silicone component present in the shampoos and conditioners according to the invention can vary widely. In certain embodiments, the first silicone component can be present in a shampoo or conditioner composition in an amount from greater than zero to about 10%, or from about 0.1% to about 5.0%, or from about 0.25% to about 2.0%, or from about 0.25% to about 0.5%, or about 0.3%, by weight based on total weight of the composition.

Optionally, the hair care compositions according to the invention can include another, e.g., the second, silicone component. Suitable second silicone components can include, but are not limited to, bis-isopropylamino-PG-propyl dimethicone/bis-isobutyl PEG-14 copolymer and/or polysorbate 20. A non-limiting example is CE 8411 which is commercially available from Dow Corning and described as a nonionic silicone emulsion with amine and polyether functionality which includes a mixture of bis-isopropylamino-PG-propyl dimethicone/bis-isobutyl PEG-14 copolymer, polysorbate 20 and butyloctanol.

The amount of the second silicone component present in the shampoos and conditioners according to the invention can vary widely. In certain embodiments, the second silicone component can be present in a shampoo or conditioner composition in an amount from greater than zero to about 10%, or from about 0.1% to about 5.0%, or from about 0.25% to about 2.0%, or from about 0.25% to about 0.5%, or about 0.3%, by weight based on total weight of the composition.

In certain embodiments wherein the hair care composition is a conditioner, both first and second silicone compounds are preferably employed.

Further, it is understood that other silicone compounds known by one having ordinary skill in the art to exhibit properties similar to the silicone compounds described herein may be used in the compositions according to the invention.

The compositions are typically in the form of a liquid.

As previously described, the fading of oxidative color or dye in hair as a result of frequent shampoo washing is a common problem for manufacturers and consumers of hair care compositions formulated for color-treated hair. There are known physical and chemical factors that influence fading of artificial dye during the hair washing and rinsing processes. In particular, it is known that fading occurs primarily through hair dye solubility in water. That is, the artificial dye that is applied to hair, e.g., the hair shaft, can be faded and/or eroded as a result of washing and rinsing the hair. It is contemplated that polymers with hydrophobically-modified and cationic functionalities are effective in preventing hair dye dissolution in water. Without intending to be bound by any particular theory, it is believed that there is a synergistic effect of the polymer's hydrophobic moieties and cationic charges that are effective to protect hair dye during shampoo washing and rinsing processes. It has been found that the prevention of color fading is improved, e.g., there is a color protection benefit, when the compositions according to the invention are employed as compared to employing known hair care compositions formulated for color-treated hair. In particular, it has been found that hair care compositions including a combination of Polyquaternium-55, quinoa and the first silicone applied to color-treated hair during the washing and rinsing processes are effective to enhance color retention.

Further, there are various components and ingredients that are generally known in the art of hair care compositions that may be included in the shampoos and conditioners according to the invention. Suitable examples include, but are not limited to, fragrances, vitamins, pH adjusters (e.g., citric acid), gelling agents and surfactants. It is contemplated that one or more of these adjuvants may also be present in the compositions of the invention in conventional amounts.

A person skilled in the art will take care to select any optional additional additives and/or amount thereof such that the advantageous properties of the compositions according to the invention are not, or are not substantially, adversely affected by the addition. These substances may be selected variously by the person skilled in the art in order to prepare a composition which has the desired properties, for example, consistency or texture.

All of the ingredients for use in preparing the compositions in accordance with the invention can be combined in any order.

### EXAMPLES

The following examples are illustrative only and are not intended to be interpreted as limiting the invention.

### Example 1—Preparation of Shampoo Formulation

A shampoo in accordance with certain embodiments of the invention was prepared by conventional mixing of the ingredients identified in Table 1 in the specified amounts.

TABLE 1

| Chemical Name | Brand Name | Percentage |
|---|---|---|
| DI Water | Water (Aqua) | 39.287% |
| Propanediol | Zemea | 1.00% |
| Cetrimonium Chloride | Cetac 30% | 0.75% |
| Polyquaternium-7 | Mackernium 007 | 0.25% |
| Polyquaternium-55 | Styleze W-10 | 1.25% |
| Superoxide Dismutase | Biocell SOD | 0.001% |
| Aloe Barbadensis (Aloe Vera) | Aloe Vera Gel | 3.10% |
| Leaf Juice | | |
| Panthenol | D, L Panthenol | 0.05% |
| Ethylene Glycol Distearate | Lexemul EGDS | 2.00% |
| PEG-50 Shea Butter | Shebu WS | 0.20% |
| Sodium C14-16 Olefin Sulfonate | Bioterge AS 40 | 30.00% |
| Cocamidopropyl Betaine | Mirataine BET | 10.00% |
| | C 30 | |
| Cocamidopropyl Hydroxysultaine | Mirataine CBS | 5.00% |
| Disodium Cocoamphodiproprionate | Mackam 2C SF | 3.00% |
| Sodium Cocoyl Glutamate | Plantapon | 1.00% |
| | ACG 50 | |
| Silicone Quaternium-16, Undeceth-11, | DC 5-7113 | 0.25% |
| Butyloctanol, Undecedth-5 | | |
| PEG-2 | Meadowquat | 0.25% |
| Dimeadowfoamaminoethylmonium | HG-70 | |
| Methosulfate | | |
| Silk Amino Acids | Crosilk Liquid | 0.10% |
| Hydrolyzed Quinoa | Quinoa Pro Ex | 0.25% |
| Benzyl Alcohol, Dehydroacetic Acid | Geogard 221 | 0.70% |
| Tocopheryl Acetate | Vitamin E | 0.01% |
| Ascorbic Acid | Vitamin C | 0.001% |
| Vitis Vinifera (Grape) | PhytoCell Tec | 0.001% |
| Fruit Cell Extract | Solar Vitis | |
| Citric Acid | Citric Acid | 0.70% |
| Fragrance | Fragrance | 0.85% |

### Example 2—Evaluation of Color Retention

The shampoo formulation of Example 1 was evaluated and compared with eight industry-leading shampoos in the professional salon market that were identified as exhibiting effective color retention. The eight industry-leading shampoos were identified as Comparative Shampoo Formulations A thru H.

Testing Procedure

Swatches of hair samples were obtained and color-treated as follows. Blonde swatches were colored with a dark intense red shade dye from a leading professional dye/color manufacturer and processed with 20-volume developer for 35 minutes.

The shampoo formulation of Example 1 and Comparative Shampoo Formulations A thru H were each used to shampoo the color-treated swatches in accordance with the following shampoo procedure.

Shampoo Procedure:

The color-treated hair swatches were shampooed 5 times, 15 times and 30 times with each of the shampoo formulations, i.e., Example 1 and Comparative Shampoo Formulations A thru H. The swatches were blow-dried and flat-ironed between each shampoo.

Data and Observations—Swatch Results

The shampooed color-treated swatches were evaluated for color retention by comparison to a standard swatch (color-treated according to the Testing Procedure above-described but not shampooed) and were rated on a scale of 0 to 5 based on the following chart.

| Rating # | Description |
|---|---|
| 0 | No fadage observed |
| 1 | Very slight fadage (98% from original color) |
| 2 | Slight fadage (95% from original color) |
| 3 | Noticeable fadage (90% from original color) |
| 4 | Average fadage (75% from original color) |
| 5 | Drastic fadage (50% from original color) |

The ratings shown below in Table 2 were assigned based on visual assessment of the shampooed color-treated swatches as compared to the non-shampooed color-treated (standard) swatch.

TABLE 2

| | After 5 | After 15 | After 30 |
|---|---|---|---|
| Formulation/Product | Shampoos | Shampoos | Shampoos |
| Shampoo of Example 1 | 1 | 1.5 | 2 |
| Shampoo A | 2 | 3 | 4.5 |
| (claims 96% color retention | | | |
| after 10 shampoos) | | | |
| Shampoo B | 2 | 2 | 5 |
| (claims 90% color retention | | | |
| after 30 washes) | | | |
| Shampoo C | 2 | 3 | 5 |
| (claims 90% color retention | | | |
| after 30 shampoos) | | | |
| Shampoo D | 1.5 | 2 | 3 |
| Shampoo E | 1 | 2 | 3 |
| Shampoo F | 1.5 | 3 | 4 |
| Shampoo G | 1.5 | 2 | 3 |
| Shampoo H | 1 | 3 | 3.5 |

The results shown in Table 2 demonstrate that based on the hair swatch testing and evaluation, the shampoo formulation of Example 1 which was prepared according to certain embodiments of the invention provided superior color protection as compared to the industry-leading Comparative Shampoo Formulations A thru H which had identified color retention properties.

It was determined that after 30 shampoos, the color-treated hair swatches shampooed with the Example 1 formulation held up to 95% of their original color applied during the color treatment process.

### Example 3—Colorimeter Test

The following three hair swatches were evaluated by an independent laboratory for color retention measurement using a colorimeter:

1. Control hair swatch of freshly color-treated hair without shampooing;

2. Color-treated hair swatch shampooed 30 times with the formulation of Example 1; and

3. Color-treated hair swatch shampooed 30 times with industry-leading Shampoo A.

A colorimeter with a 0.5″ light port was employed and the measurements shown below in Table 3 were taken.

TABLE 3

| | | | | Change in l, a, b values | | | |
|---|---|---|---|---|---|---|---|
| Colorimeter Results | | | | compared to Control | | | |
| Sample | l | a | b | Δl | Δa | Δb | ΔE |
| Control | 29.27 | 28.65 | 17.67 | — | — | — | — |
| Example 1 | 30.82 | 31.51 | 19.46 | 1.55 | 2.86 | 1.79 | 3.7 |
| Comparative | 37.17 | 29.89 | 18.84 | 7.9 | 1.24 | 1.17 | 8.08 |
| Shampoo A | | | | | | | |

wherein:
l = lightness;
a = red and green;
b = blue and yellow; and
0 = day 0.

### Example 4—Preparation of Conditioner Formulation

A conditioner formulation in accordance with certain embodiments of the invention was prepared by conventional mixing of the ingredients identified in Table 4 in the specified amounts.

TABLE 4

| Chemical Name | Trade Name | Percentage |
|---|---|---|
| DI Water | Water (Aqua) | 82.96% |
| Propanediol | Zemea | 0.99% |
| Guar Hydroxypropyltrimonium | Jaguar C-17 | 0.15% |
| Chloride | | |
| Citric Acid | Citric Acid | 0.08% |
| Polyquaternium-7 | Mackernium 007 | 0.25% |
| Polyquaternium-55 | Styleze W-10 | 1.00% |
| Panthenol | D, L Panthenol | 0.05% |
| Superoxide Dismutase | Biocell SOD | 0.001% |
| Cetrimonium Chloride | Cetac 30% | 1.80% |
| Stearyl Alcohol | CO-1895 Fatty Alcohol | 1.50% |
| Cetyl Alcohol | CO-1695 Fatty Alcohol | 1.50% |
| Behentrimonium Methosulfate and | | |
| Cetearyl Alcohol | Incroquat Behenyl TMS | 3.20% |
| Isopropyl Myristate | Lipo IPM | 0.75% |
| Sweet Almond Oil | Sweeat Almond Oil | 0.50% |
| Cocos Nucifera (Coconut) Oil | Coconut Oil 76 | 0.001% |
| Sunflower Seed Oil | Sunflower Seed Oil | 0.25% |
| Oryza Sativa (Rice) Bran Oil | Rice Bran Oil | 0.01% |
| Hydrolyzed Silk | Crosilk 1000 | 0.001% |
| Hydrolyzed Quinoa | Quinoa Pro Ex | 0.50% |
| Quaternium-95, Propanediol | Chromaveil | 0.05% |
| Bis-Diisopropanolamino-PG- | CE 8411 | 1.00% |
| propyl Dimethicone/Bis-Isobutyl | | |
| PEG-14 Copolymer | | |
| Silicone Quaternium-16, | DC 5-7113 | 2.00% |
| Undeceth-11, Butyloctanol, | | |
| Undeceth-5 | | |
| Benzyl Alcohol, Dehydroacetic | Geogard 221 | 0.70% |
| Acid | | |
| Ascorbic Acid | Vitamin C | 0.001% |
| Tocopheryl Acetate | Vitamin E | 0.001% |
| Vitis Vinifera (Grape) | PhytoCell Tec | 0.001% |
| Fruit Cell Extract | Solar Vitis | |
| Fragrance | Fragrance | 0.75% |

The invention is well adapted to carry out the objects and attain the ends and advantages mentioned, as well as others inherent therein. While the invention has been depicted and described and is defined by reference to certain embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described embodiments of the invention are exemplary only and are not exhaustive of the scope of the invention. Consequently, the invention is intended to be limited only by the spirit and scope of the appended claims, giving full cognizance to equivalents in all respects.

What is claimed is:

1. A hair care composition for color-treated hair, comprising:
a cationic terpolymer of vinylpyrrolidone, dimethylaminopropyl methacrylamide, and methacryloylaminopropyl lauryldimonium chloride;
quinoa; and
a first silicone.

2. The hair care composition of claim 1, wherein the color-treated hair has applied thereto an artificial dye and wherein the hair care composition is effective to at least partially prevent the artificial dye from fading and washing out.

3. The hair care composition of claim 1, wherein said composition is selected from the group consisting of shampoo and conditioner.

4. The hair care composition of claim 1, wherein the first silicone comprises an anionic silicone.

5. The hair care composition of claim 1, wherein the first silicone is selected from the group consisting of Silicone Quaternium-16, Undeceth-5, Undeceth-11, butyloctanol, and mixtures thereof.

6. The hair care composition of claim 1, further comprising a second silicone.

7. The hair care composition of claim 6, wherein the second silicone comprises silicone amine.

8. The hair care composition of claim 7, wherein the second silicone comprises bis-isopropylamino-PG-propyl dimethicone/bis-isobutyl PEG-14 copolymer.

9. The hair care composition of claim 7, wherein the second silicone comprises a mixture of bis-isopropylamino-PG-propyl dimethicone/bis-isobutyl PEG-14 copolymer, polysorbate 20 and butyloctanol.

10. The hair care composition of claim 6, wherein the first silicone compound comprises a mixture of Silicone Quaternium-16, Undeceth-11, butyloctanol, Undeceth-5, and the second silicone compound comprises a mixture of bis-isopropylamino-PG-propyl dimethicone/bis-isobutyl PEG-14 copolymer, polysorbate 20, butyloctanol.

11. A method of preparing a hair care composition for color-treated hair, the composition having improved color retention, comprising:
preparing a mixture, comprising:
a cationic terpolymer of vinylpyrrolidone, dimethylaminopropyl methacrylamide, and methacryloylaminopropyl lauryldimonium chloride;
quinoa; and
a first silicone.

12. A method of improving the retention of artificial color for color-treated hair during washing and rinsing of the color-treated hair, comprising:
preparing a composition, comprising:
a cationic terpolymer of vinylpyrrolidone, dimethylaminopropyl methacrylamide, and methacryloylaminopropyl lauryldimonium chloride;
quinoa; and
a first silicone;
wetting the color-treated hair;
applying the composition to wetted color-treated hair; and
following applying the composition, rinsing with water to at least partially remove the composition from the color-treated hair
